(19) United States
(12) Patent Application Publication
(10) Pub. No.: US 2011/0236354 A1
(43) Pub. Date: Sep. 29, 2011

(54) Symbiotic bacterial oncolysis-a beacon-based method for enhanced destruction of solid tumors-including both the tumor core and rim

(76) Inventor: Gerhard B. Holt, Sydney (AU)

(21) Appl. No.: 12/661,692

(22) Filed: Mar. 23, 2010

(51) Int. Cl. A61K 35/74; A61P 35/00

(52) U.S. Cl. 424/93.4

(57) ABSTRACT

Symbiotic bacterial oncolysis is a method to directly lyse and/or otherwise destroy the tumor core and rim of solid tumors using two (or more) phases of treatment. The first phase localizes with specificity for features of tumor microenvironment. The second (and/or subsequent) phase(s)—otherwise known as the SYMBIOT—localizes with specificity for features—designed, selected or inherent—of an earlier treatment phase (typically the phase 1). This earlier phase (typically phase 1) acts as a BEACON to help improve the specificity and/or localization of the subsequent—SYMBIOT—phases of treatment. Overall this should lead to improved solid tumor destruction relative to current art.

### MOTIVATION AND PRIOR ART

It is well known that the cores of solid tumors tend to be underperfused and therefore hypoxic.

This is largely due to the rapid growth of tumor cells on the rim of the solid tumor outpacing the growth of “supportive” tumor vasculature. The cores of solid tumors tend to develop a characteristic tumor microenvironment characterized by many features including hypoxia, acidity and many tumor cells in a quiescent or partially quiescent state.

Traditional cancer treatments like chemotherapy and radiotherapy have diminished effectiveness in the hypoperfused cores of solid tumors. Radiation therapy depends on generation of oxygen free radicals for much of its effect. [1] Chemotherapy is often more effective on rapidly dividing non-quiescent cells, and needs to get to the tumor cells via the bloodstream to work. The tumor core therefore is thought to be a source of development of tumor resistance to treatment and of eventual recurrence.

Direct bacterial oncolysis offers a highly robust approach to destroying tumor cells.

Clostridia—a type of strict anaerobic spore forming bacteria—has been known for many decades to be effective at invading, colonizing and lysing the hypoxic underperfused cores of solid tumors.[2] Clostridium Novyi was found to be particularly effective at direct bacterial oncolysis, so a genetically modified version with no toxin—Clostridium Novyi NT was developed. [3-4]

Many other types of bacteria have also been tried for direct bacterial oncolysis, and to locally produce, activate, enhance, or otherwise help selectively deliver effective levels of medications, cytokines, and/or other therapeutic substances, and/or as a vector for altered protein expression, pro-drug conversion and immune system activation among other approaches.

Clostridium Novyi NT was quite effective a lysing tumor core, but—since it is a strict anaerobe—it spares the well perfused tumor rim. This led to a wide variety of combination treatment approaches including use of additional chemotherapy and/or radiotherapy, RAIT—use of radioimmunoconjugate monoclonal antibodies targeting tumor antigens (CEA), genetic modification approaches to release prodrug converting enzymes, cytokines, and lyposomase to more selectively deliver chemotherapeutic agents encapsulated in liposomes. [2, 5-7, 10-11]

Clostridium Novyi NT has many advantages see [10][11] for reviews). One of clostridium novyi NT's main advantages is its high tumor specificity—but being a strict anaerobe—it leaves the perfused tumor rim.

This suggests the use of a second lysing bacteria—an aerobe or a facultative anaerobe to lyse the rim. One key problem with this approach is that the rim lysing bacteria may not be sufficiently specific for the tumor tissue and may cause potentially dangerous systemic infection.

### METHOD

### Phase 1

Use a bacteria with specificity for the tumor core microenvironment—such as Clostridium Novyi NT (by way of example but not by way of limitation)—to initially infect and lyse the tumor core.

### Phase 2

Use features of this tumor core infecting bacteria (Phase 1) to more effectively and specifically localize a second phase of treatment with enhanced specificity to the area around the tumor core including the tumor rim. This will be referred to as Phase 2 of treatment or as the SYMBIOT.

The specificity of strict anaerobes for the tumor core microenvironment could be used as a beacon that could subsequently selectively localize other symbiotic treatment modalities. These modalities would then selectively target the region proximal to this Phase 1—including the tumor rim—without depending any longer on direct specificity to the tumor microenvironment.

In other words, this Phase 2 need not be dependent on the general tumor rim microenvironment but rather could be dependent on specific substance(s) intentionally provided in a symbiotic manner by modifications on the Phase 1 treatment.

By way of example, not by way of limitation, the Phase 2 could be an aerobic or facultative anaerobic bacteria including specifically modified or selected bacteria which could withstand conditions in the tumor rim. Various types of bacteria can be used for the Phase 2. The specific choice of Phase 2 bacteria is to be made with consideration for and weighing several preferred attributes and goals including—by way of example, not by way of limitation—that it should:

1) be excellent at colonizing and at lysing cells (even in the presence of a strong neutrophil response).

2) be exquisitely sensitive to many different antibiotics &/or have multiple defects that will allow it's range to be safely and specifically restricted to the area near the Phase 1. (see below)

3) have low systemic toxicity to reduce the chance of shock or other severe problems. The Phase 1 treatment acts as a “BEACON” to more specifically localize the Phase 2 treatment/SYMBIOT.

This can occur in several ways—by way of example not by way of limitation—including:

1) Having the Phase 1 secrete substances that will protect itself and that will locally protect the Symbiot (Phase 2) against a systemic substance or substances which would otherwise destroy, deactivate or eliminate them. By way of example, not by way of limitation, the Phase 1 could produce beta-lactamase (or other suitable antibiotic inactivating substances) which would protect the Phase 1 and nearby Phase 2 bacteria from systemic administration of beta-lactam antibiotics (or other suitable selected antibiotics) that would destroy such bacteria elsewhere in the body.

2) Having the Phase 1 secrete substances that will facilitate the local growth and survival of an intentionally auxotrophic or bradytrophic Phase 2 Symbiot.

3) Using monoclonal antibody (or multispecific antibody)-based treatments directed against highly specific antigens/epitopes on the surface of the Phase 1 bacteria (&/or Phase 2 if additional phases are selected), including by way of example, not by way of limitation—radioimmunoconjugates, minigels/EBV-based treatment and or liposomal encapsulation based treatment for specific and/or higher dose delivery of—by way of example, not by way of limitation—traditional chemotherapeutic drugs, prodrug converting enzymes, prodrugs, cytokines or other therapeutic substances.

4) Having Phase 1 locally dampen the immune response which would facilitate the proximal survival and growth of the Phase 2 symbiot.

### Optional Additional Phases

By the same principal, additional phases can also use Phase 2 as a “BEACON” to more specifically localize to the area at or near Phase 2.

### REFERENCES

(1) Overcoming the hypoxic barrier to radiation therapy with anaerobic bacteria. Bettegowda C, Dang L H, Abrams R, Huso D L, Dillehay L, Cheong I, Agrawal N, Borzillary S, McCaffery J M, Watson E L, Lin K S, Bunz F, Baidbo K, Pomper M G, Kinzler K W, Vogelstein B, Zhou S.
Proc Natl Acad Sci USA. 2003 Dec. 9; 100(25):15083-8. Epub 2003 Dec. 1. PMID: 14657371
(2) The use of clostridial spores for cancer treatment.
Barbé S, Van Mellaert L, Anné J.
J Appl Microbiol. 2006 September; 101(3):571-8. Review. PMID: 16907807
(3) Combination bacteriolytic therapy for the treatment of experimental tumors.
Dang L H, Bettegowda C, Huso D L, Kinzler K W, Vogelstein B.
Proc Natl Acad Sci USA. 2001 Dec. 18; 98(26):15155-60. Epub 2001 Nov. 27. PMID: 11724950
(4) Bacteriolytic therapy can generate a potent immune response against experimental tumors. Agrawal N, Bettegowda C, Cheong I, Geschwind J F, Drake C G, Hipkiss E L, Tatsumi M, Dang L H, Diaz L A Jr, Pamper M, Abusedera M, Wahl R L, Kinzler K W, Thou S, Huso D L, Vogelstein B. Proc Natl Acad Sci USA. 2004 Oct. 19; 101(42): 5172-7. Epub 2004 Oct. 7. PMID: 15471990
(5) Targeting cancer with bugs and liposomes: ready, aim, fire.
Cheong I, Huang X, Thornton K, Diaz L A Jr, Zhb S.
Cancer Res. 2007 Oct. 15; 67(20):9605-8. Review. PMID: 17942887
(6) A bacterial protein enhances the release and efficacy of liposomal cancer drugs. Cheong I, Huang X, Bettegowda C, Diaz L A Jr, Kinzler K W, Zhou S, Vogelstein B. Science. 2006 Nov. 24; 314(5803):1308-11. PMID: 17124324
(7) Optimized clostridium-directed enzyme prodrug therapy improves the antitumor activity of the novel DNA cross-linking agent PR-104.
Liu S C, Ahn G O, Kioi M, Dorie M J, Patterson A V, Brown J M.
Cancer Res. 2008 Oct. 1; 68(19):7995-8003. PMID: 18829557
(8) Containment of tumor-colonizing bacteria by host neutrophils.
Westphal K, Leschner S, Jablonska J, Loessne, Weiss S.
Cancer Res. 2008 Apr. 15; 68(8):2952-60. PMID: 18413765
(9) Targeted therapy with a Salmonella typhimurium leucine-arginine auxotroph cures orthotopic human breast tumors in nude mice.
Zhao M, Yang M, Ma H, LI X, Tan X, Li S, Yand Z, Hoffman R M.
Cancer Res. 2006 Aug. 1; 66(15):7647-52. PMID: 16885365
(10) Bacterial therapies: completing the cancer treatment toolbox.
St Jean A T, Zhang M, Forbes N S.
Curr Opin Biotechnol. 2008 October; 19(5):511-7. Epub 2008 Sep. 18.
Review. PMID: 18760353
(11) Clostridial spores as live ‘Trojan horse’ vectors for cancer gene therapy: comparison with viral delivery systems. Wei M Q. Ren R, Good D, Anné J. Genet Vaccines Ther. 2008 Feb. 17; 6:8. PMID: 18279524

1) Symbiotic bacterial oncolysis is a method to directly lyse and/or otherwise destroy the tumor core and rim.

2) Phase 1 (&/or subsequent phases) can be used as a beacon to more selectively and specifically localize the symbiotic treatment(s) to the area near the tumor core—including the tumor rim—providing more complete treatment than current art.

3) Modified and/or selected bacteria and/or other treatments can be given with fewer systemic risks and/or at effectively higher dosage at the target site due to the enhanced specificity of this method.
